(19) United States
(12) Patent Application Publication
(10) Pub. No.: US 2022/0387076 A1
(43) Pub. Date: Dec. 8, 2022

(54) BLADE HEAD FOR A DERMAPLANING EXFOLIATION TOOL

(71) Applicant: SPA BENJAMIN LLC, LOS ANGELES, CA (US)

(72) Inventor: IAN MACCOLL, MILL VALLEY, CA (US)

(73) Assignee: SPA BENJAMIN LLC, LOS ANGELES, CA (US)

(21) Appl. No.: 17/804,646

(22) Filed: May 31, 2022

Related U.S. Application Data
(60) Provisional application No. 63/208,149, filed on Jun. 8, 2021.

(51) Int. Cl. A61B 17/54; A61B 17/3205

(52) U.S. Cl. CPC A61B 17/54; A61B 17/3205; A61B 2017/00761

(57) ABSTRACT

In a tool, a blade head configured to releasably connect to a handle includes a first molded article; and a second molded article that interfaces the first molded article; wherein the second molded article includes: a connector end portion that is integrated within the second molded article so that the connector end portion is seamless; the connector end portion has a channel therein; and the channel is configured to releasably connect about the handle.

### CROSS-REFERENCE TO RELATED APPLICATIONS

This application claims priority to and the benefit of U.S. provisional patent application No. 63/208,149, filed Jun. 8, 2021, all of which is incorporated herein in its entirety.

### BACKGROUND OF THE DISCLOSURE

The present disclosure generally relates to apparatus for dermaplaning and, more particularly, to a dermaplaning tool that minimizes breakage of the head portion from the handle portion.

As shown in prior art FIGS. 1-3, the Applicant herein previously designed a dermaplaning exfoliation tool 10 to remove dead skin and peach fuzz with its single blade 50 to deliver a smooth complexion, even skin tone, and clear pore congestion. Dermaplaning enhances product penetration, helps makeup to go on smoother, and reduces the appearance of lines.

As shown, the tool 10 includes an aluminum handle 30 to optimize user control. The top end of the handle 30 includes a post 35 that is captured within the channel 46 between two halves 42a, 42b of the blade sheath or head 40, when installing the blade head 40 on to the post 35.

In practice, it has been determined that the two-piece head construction around the post 35 causes a weak point 40a (FIG. 2) at or near a seam 40b (FIG. 3), and that after extended use, or upon multiple on/off evolutions for blade replacement, the head 40 cracks at the bottom thereof.

As can be seen, there is a need for an improved head portion of a dermaplaning tool which minimizes breakage of the head portion.

### SUMMARY OF THE DISCLOSURE

In one aspect of the present disclosure, in a tool, a blade head configured to releasably connect to a handle 160 comprises a first molded article; and a second molded article that interfaces the first molded article; wherein the second molded article includes: a connector end portion that is integrated within the second molded article so that the connector end portion is seamless; the connector end portion has a channel therein; and the channel is configured to releasably connect about the handle.

In another aspect of the present disclosure, in a tool, a blade head configured to releasably connect to a handle comprises a blade head portion that is a first single molded article and is a part of a second single molded article; a connector end portion that is another part of the second single molded article; wherein the connector end portion is seamless; wherein the connector end portion has a channel therein; and wherein the channel is configured to releasably connect about the handle.

In a further aspect of the present disclosure, in a dermaplaning tool, a blade head configured to releasably connect to a handle comprises one seamless molded article is a part of a blade end portion and an entire connector end portion with a channel therein; wherein the channel is configured to releasably connect about the handle.

These and other features, aspects and advantages of the present disclosure will become better understood with reference to the following drawings, description, and claims.

### BRIEF DESCRIPTION OF THE DRAWINGS

FIG. 1 is a side elevation view of a prior art tool developed by the Applicant herein.

FIG. 2 is a close-up view to show a weak point at a connection between a blade head and a handle of FIG. 1.

FIG. 3 is an end view of the blade head of FIG. 1 to show the two blade head halves and weak point.

FIG. 4 is a perspective, exploded view of an exemplary dermaplaning tool according to the present disclosure.

FIG. 5 is a perspective view of an exemplary blade head of a dermaplaning tool according to the present disclosure.

FIG. 6A is a perspective view of one exemplary molded article of a blade head according to the present disclosure.

FIG. 6B is a perspective view of another exemplary molded article of a blade head according to the present disclosure.

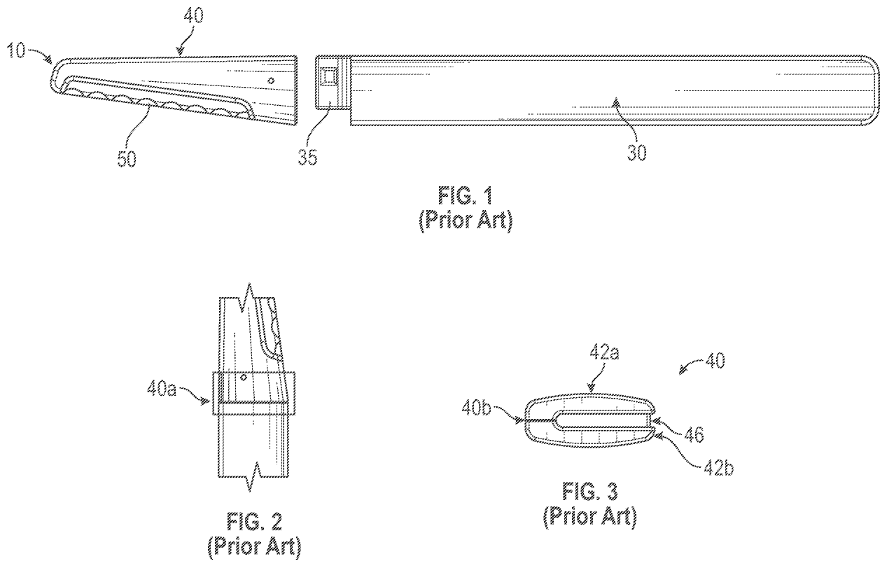

### DETAILED DESCRIPTION OF THE DISCLOSURE

In the following description, certain specific details are set forth in order to provide a thorough understanding of various example embodiments of the disclosure. However, one skilled in the art will understand that the disclosure may be practiced without these specific details. In other instances, well-known structures associated with manufacturing techniques have not been described in detail to avoid unnecessarily obscuring the descriptions of the example embodiments of the present disclosure.

The following detailed description is of the best currently contemplated modes of carrying out the disclosure. The description is not to be taken in a limiting sense, but it is merely for the purpose of illustrating the general principles of the disclosure, since the scope of the disclosure is best defined by the appended claims.

Various inventive features are described below that can each be used independently of one another or in combination with other features. However, any single inventive feature may not address any of the problems discussed above or may only address one of the problems discussed above. Further, one or more of the problems discussed above may not be fully addressed by any of the features described below.

Unless the context requires otherwise, throughout the specification and claims that follow, the word “comprise” and variations thereof, such as “comprises” and “comprising,” are to be construed in an open, inclusive sense, that is, as “including, but not limited to.”

As used herein, the terms “embodiment” and “embodiments” are intended to be used interchangeably. In other words, the singular includes the plural, and vice versa.

Here, the technical problem to be solved is that a blade head for a dermaplaning tool can have a tendency of cracking or breaking after multiple attachments and detachments from a handle of the tool.

Broadly, the present disclosure solves the foregoing problem by a connector end portion of a blade head that is molded as a single, integral piece and therefore seamless. The present disclosure further provides a connector of a blade head that can securely hold a handle of the dermaplaning tool and easily release the handle as desired. These features can minimize or eliminate the tendency in past designs which have led to cracking or breaking the connector of the blade head.

Generally, and according to an embodiment of the present disclosure, a dermaplaning tool can include a single-molded, four-sided, walled connector with thickened portions on the blade edge side of the blade sheath or head to provide a robust connection between a post of a handle and the blade head, the lower end of which can be fixedly attached around an upper circumferential periphery of the connector. The connector interior geometry can be configured to receive the post in a channel.

Also generally, and in an embodiment of the present disclosure, within the channel of the connector, there may be detents that mirror up and engage corresponding detents on the face sides of the post. According to embodiments of the present disclosure, the blade may include a blade guard, and there can be a plastic cover for placement over the blade for storage that includes a thickened wall on the inside facing surface to the blade edge.

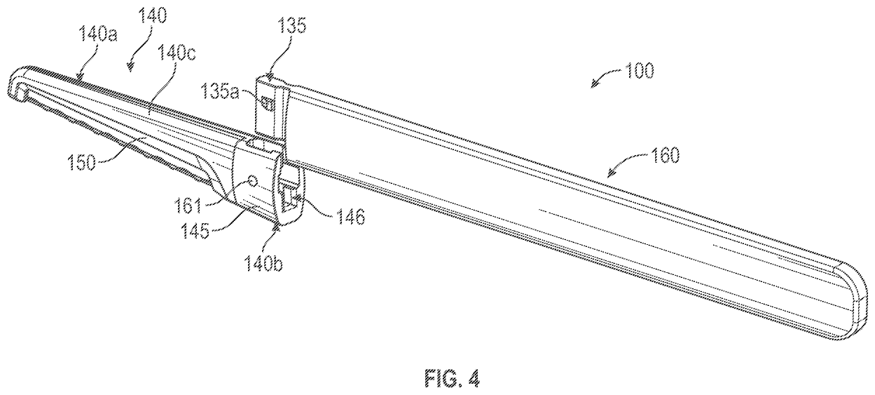

More specifically, and referring to FIG. 4, in an embodiment of the present disclosure, a dermaplaning tool 100 may include a blade head 140 and a handle 160. In an embodiment of the disclosure, the blade head 140 and the handle 160 may extend along a longitudinal axis of the tool 100. The blade head 140 and the handle 160 may be configured to releasably connect to and releasably disconnect from one another, according to an embodiment of the present disclosure. In an aspect of the present disclosure, the blade head 140 and the handle 160 may connect to and disconnect from one another by their relative movement in a direction generally perpendicular to the longitudinal axis.

In an embodiment of the present disclosure, blade head 140 may be configured to hold a blade 150 therein. The handle 160 may be configured to enable a user to hold the tool 100 in the user's hand. The handle 160 may include, at an end thereof, a post 135. In an embodiment of the present disclosure, the post 135 may be configured to be inserted into the blade head 140. Further, the post 135 may releasably connect and disconnect from the blade head 140, in an embodiment of the present disclosure.

According to the present disclosure, the handle 160 may include one or more post detents 135a on the post 135. In an aspect of the present disclosure, a pair of post detent 135a may be on opposed sides of the post 135. The one or more post detents 135a may be configured to mate, such as by friction fit, with one or more blade head detents 146a which may be on opposed interior parts of the blade head 140.

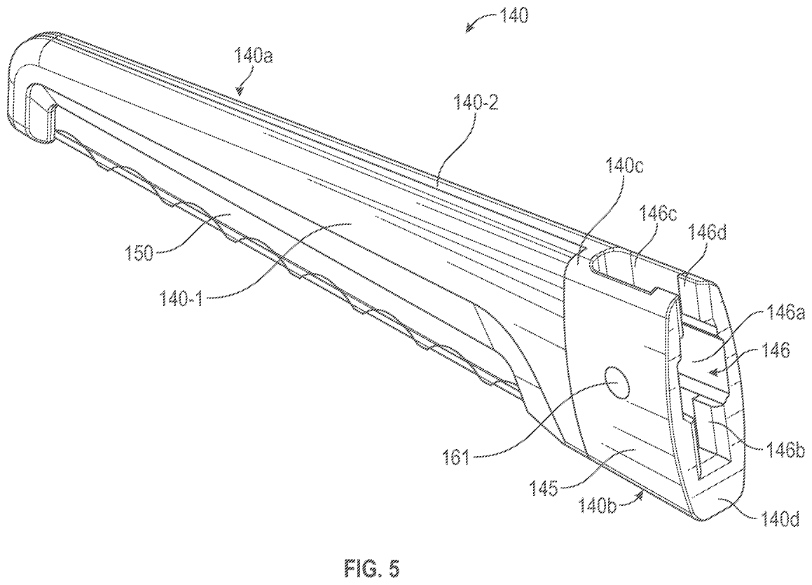

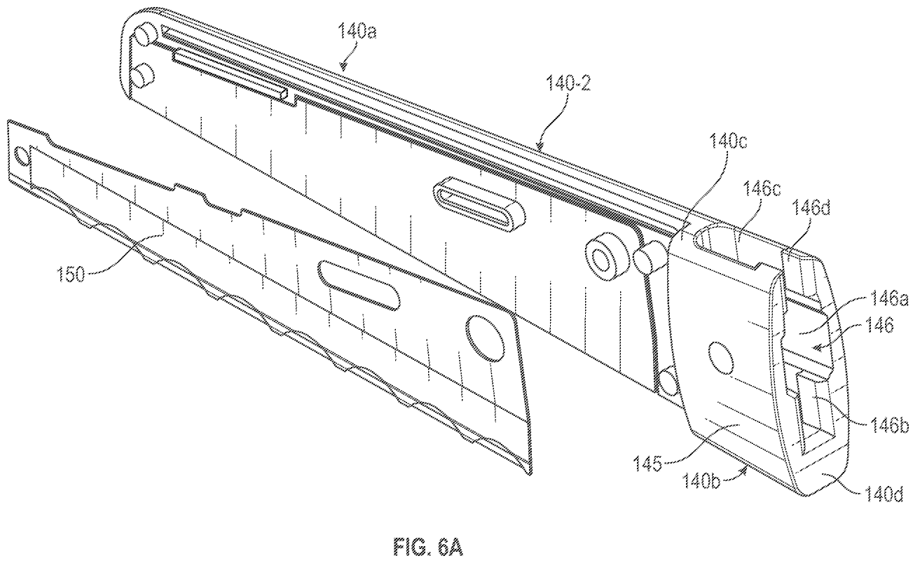

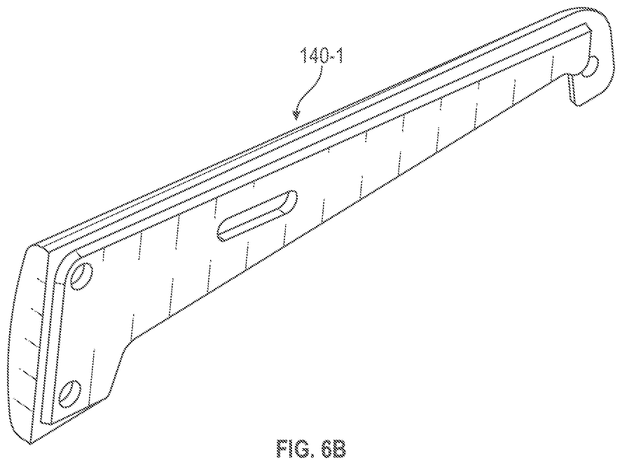

In FIGS. 5, 6A and 6B, according to an embodiment of the present disclosure, the blade head 140 may include a first molded article 140-1 and a second molded article 140-2 that interfaces the former. In embodiments of the present disclosure, one or both the first and second molded articles 140-1, 140-2 can be single molded articles, and one or both of which can be seamless. In other words, the articles may be individually molded as a single piece, rather than multiple pieces, to eliminate any seams where the pieces are otherwise joined.

In FIGS. 6A and 6B, in an embodiment of the present disclosure, the first and second molded articles 140-1, 140-2 may be configured to hold therebetween the blade 150. For example, one or both the articles 140-1, 140-2 may include apertures and/or projections that may mate with one another, and/or mate with apertures in the blade 150. In other embodiments, the first and second molded articles 140-1, 140-2 may be attached/adhered to one another, such as by welding.

In FIGS. 5 and 6A, according to an embodiment of the present disclosure, the blade head 140 may include, at one end thereof, a blade head portion 140a. At an opposite end thereof, the blade head 140 may include a connector end portion 140b. In an embodiment, the connector end portion 140b can directly interface the blade end portion 140a. In an embodiment, the connector end portion 140b may be a single molded, four-sided wall article.

According to the present disclosure, the blade end portion 140a can be/comprise/is the entire first molded article 140-1 and a part of the second molded article 140-2. In an embodiment, the connector end portion 140b can be/comprise/is another part of the second molded article 140-2. In other words, in an embodiment, the second molded article 140-2 is a part of the blade end portion 140a and is also the entire connector end portion 140b. Thereby, the connector end portion 140b can be wholly integrated within the second molded article 140-2 so that the connector end portion 140b is seamless.

In embodiments, the blade head portion 140a can consist of the first single molded article 140-1, a part of the second single molded article 140-2, and the blade 150. In embodiments, the connector end portion 140b can consist of another part of the second single molded article 140-2.

In an embodiment, the connector end portion 140b may be configured to releasably connect with the handle 160, such as with the post 135. In an embodiment, the connector end portion 140b may have a top surface or wall 140c that may be configured to receive therethrough the handle 160. In embodiments, the connector end portion 140b may have an end surface or wall 140d that may be configured to receive therein the handle 160.

In an embodiment, the connector end portion 140b may have a connector 145. According to an aspect of the disclosure, the connector 145 can be seamless. In an embodiment, the connector 145 may have channel 146 therein. In an embodiment, the connector 145 and, thus the channel 146, may be configured to releasably connect about the handle 160, such as about the post 135. In an embodiment, the channel 146 may have a configuration that mirrors the configuration of the post 135.

In FIGS. 5 and 6A, according to an embodiment, the channel 146 may extend through a top surface or wall 140c of the blade head 140 that includes the connector end portion 140b. In an embodiment, the channel 146 may extend through an end surface or wall 140d of the connector end portion 140b. In an embodiment, the channel 146 may be one continuous opening between the top surface 140c and the end surface 140d.

In embodiments, the channel 146 may include a top opening in the top surface 140c. In an embodiment, the top opening may include one or more sub-openings 146c, 146d. For example, the sub-openings 146c, 146d may have the same or different configurations. The configurations of the sub-openings 146c, 146d may mirror a bottom and sides of the post 135, in embodiments. In other embodiments, the sub-openings 146c, 146d may form a single continuous top opening.

In embodiments, the channel 146 may include an end opening in the end surface 140d. In an embodiment, the end opening may include one or more sub-openings/detents 146a, 146b. For example, the sub-openings/detents 146a, 146b may have the same or different configurations. The configurations of the sub-openings/detents 146a, 146b may mirror sides of the post 135, such as the post detents 135a, in embodiments. In other embodiments, the sub-openings/detents 146a, 146b may form a single continuous end opening.

From the foregoing, and as depicted in FIG. 4, it can therefore be understood that the end opening 146a, 146b and the top opening 146c, 146d are configured to enable the post 135 to be inserted into the top opening and moved in a downwardly direction (from the viewpoint of FIG. 4) into the channel 146 and into the end opening, according to an embodiment.

In FIGS. 5 and 6A, one or more of the detents 146a mentioned above can be within the connector end portion 140b, in an embodiment. The one or more detents 146a may further extend into the channel 146, in an embodiment. The one or more detents 146a may be configured to releasably engage the handle 160, such as at the post 135, in an embodiment. Further, the one or more detents 146a may be configured to engage the one or more detents 135a on the post 135.

In FIGS. 5 and 6A, the connector end portion 140b may also include detents 161, such as on opposing exterior surfaces thereof, in an embodiment. The detents 161 may be configured to mate to a cover (not shown) which can be configured to cover and uncover the blade head portion 140.

The foregoing discussion of the present disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights which include alternative embodiments, configurations, or aspects to the extent permitted, including alternate, interchangeable and/or equivalent structures to those claimed, whether or not such alternate, interchangeable and/or equivalent structures are disclosed herein, and without intending to publicly dedicate any patentable subject matter.

We claim:

1. In a tool, a blade head configured to releasably connect to a handle, comprising:
a first molded article; and
a second molded article that interfaces the first molded article;
wherein the second molded article includes:
a connector end portion that is integrated within the second molded article so that the connector end portion is seamless;
the connector end portion has a channel therein; and
the channel is configured to releasably connect about the handle.

2. The blade head of claim 1, wherein:
the first and second molded articles are adhered to one another.

3. The blade head of claim 1, wherein:
the first and second molded articles are each single molded articles.

4. The blade head of claim 1, further comprising:
a blade end portion that interfaces the connector end portion;
wherein the blade end portion comprises the first molded article and a part of the second molded article; and
wherein the connector end portion comprises another part of the second molded article.

5. The blade head of claim 1, wherein:
the channel extends through a top surface of the connector end portion.

6. The blade head of claim 1, wherein:
the channel extends through an end surface of the connector end portion.

7. The blade head of claim 1, wherein:
the channel includes a pair of detents that are configured to releasably engage the handle.

8. In a tool, a blade head configured to releasably connect to a handle, comprising:
a blade head portion that is a first single molded article and is a part of a second single molded article;
a connector end portion that is another part of the second single molded article;
wherein the connector end portion is seamless;
wherein the connector end portion has a channel therein; and
wherein the channel is configured to releasably connect about the handle.

9. The blade head of claim 8, wherein:
the blade head portion consists of:
the first single molded article;
the part of the second single molded article; and
a blade.

10. The blade head of claim 8, wherein:
the connector end portion consists of the another part of the second single molded article.

11. The blade head of claim 8, wherein:
the channel is one continuous opening between a top surface and an end surface of the connector end portion.

12. The blade head of claim 8, wherein:
the channel includes a top opening in a top surface of the connector end portion.

13. The blade head of claim 8, wherein:
the channel includes an end opening in an end surface of the connector end portion.

14. In a dermaplaning tool, a blade head configured to releasably connect to a handle, comprising:
one seamless molded article is a part of a blade end portion and an entire connector end portion with a channel therein;
wherein the channel is configured to releasably connect about the handle.

15. The blade head of claim 14, further comprising:
another seamless molded article is another part of the blade end portion and configured to mate with the one seamless molded article.

16. The blade head of claim 14, wherein:
the connector end portion has an end surface configured to receive therein the handle.

17. The blade head of claim 14, wherein:
the connector end portion has a top surface configured to receive therethrough.

18. The blade head of claim 14, wherein:
the connector end portion is configured to receive and release the handle in a direction that is generally perpendicular to a longitudinal axis of the dermaplaning tool.

19. The blade head of claim 14, wherein:
the connector end portion is a single molded, four-sided walled article.
